(19) United States
(12) Patent Application Publication
(10) Pub. No.: US 2023/0200698 A1
(43) Pub. Date: Jun. 29, 2023

(54) MENTAL HEALTH ASSISTANT DEVICE AND METHODS OF TRAINING AND USING THE SAME FOR AMELIORATING AUDIBLE DISTRESS IN AN AMBIENT ENVIRONMENT

(71) Applicants: Chaya Beck, Superior, CO (US); Kirk Patrick Miller, Superior, CO (US)

(72) Inventors: Chaya Beck, Superior, CO (US); Kirk Patrick Miller, Superior, CO (US)

(21) Appl. No.: 18/146,352

(22) Filed: Dec. 23, 2022

Related U.S. Application Data
(60) Provisional application No. 63/266,002, filed on Dec. 24, 2021.

(51) Int. Cl. A61B 5/16; A61B 5/00; G16H 20/70; G16H 50/20

(52) U.S. Cl. CPC A61B 5/165; A61B 5/4803; G16H 20/70; G16H 50/20; A61B 2560/0242

(57) ABSTRACT

A method for improving the mental health of children, families and homes using “positive feedback loop” technology is described. One or more aspects of the method include obtaining a device; training the device to assign a predetermined threshold to at least one input, where the at least one input comprises a measurement of at least one condition of an ambient environment; measuring the at least one input; assigning a score to the measurement of the at least one condition of an ambient environment; evaluating whether the score exceeds the predetermined threshold; alerting at least one user when the score exceeds the predetermined threshold; and repeating the measuring, assigning, and evaluating steps until the score exceeds the predetermined threshold when the score does not exceed the predetermined threshold.

### RELATED APPLICATIONS

The present application claims the benefit of U.S. Provisional Patent Application No. 63/266,002, filed on Dec. 24, 2021, which is incorporated herein by reference in its entirety for all purposes.

### FIELD

The following relates generally to mental health, and more specifically to devices and methods for improving the mental health of children, families, and homes utilizing “positive feedback loop” technology.

### BACKGROUND OF TECHNICAL PROBLEM

We are currently undergoing a global mental health crisis. It is estimated that one in four individuals suffer from mental illness, addition, or trauma. This crisis can take a toll on individuals and families.

Making matters worse, over the last decade, there has been a proliferation of intelligent devices that allow individuals to access all the information in world history simply by using their phones. While intelligent devices, such as but not limited to “smart phones” have had a profoundly positive impact on our lives in many ways, we are now learning that these devices can cause harm. For instance, social media applications use addictive feedback-loop technologies to drive up engagement. For instance, employees of at least one social media platform have admitted that the platform's algorithm has been optimized to display a disproportionate amount of rage-provoking content.

On the other hand, technologies that promote a “positive feedback-loop” for mental health are scarce. Part of the challenge for creating a device that is configured to promote positive mental health, is that positive emotions do not promote as strong of an emotional response as compared to negative emotions. Thus, positive emotional responses are relatively more difficult to detect by devices. In addition, while viewing “negative” electronic content can ruin an otherwise good day for most people, the opposite is less true. One can view positive content, but still be upset due to some other environmental trigger that is not ordinarily detected by a device. Accordingly, while it is easy for a device to pull a user into a cycle of negatively provocative content, it is difficult to engineer a device that can pull the user into a positive mental feedback-loop. Consequently, methods, systems, and devices that are programmed to promote a positive mental response from at least one user are needed.

Not only electronic devices affect mental responses, however. The social environment also contains many stressors that current devices are ill equipped to ameliorate.

For instance, devices currently have a difficult time quantifying and aggregating levels of environmental distress in such a way that these distress signals can be converted into useful information.

Accordingly, there is also a need for a device that is configured to convert audible distress signals into useful information that can trigger the device to perform an action to ameliorate the distress, without necessarily requiring user input.

### SUMMARY OF TECHNICAL SOLUTION

To solve at least the above technical problems, a method, non-transitory computer-readable medium, system, and apparatus programed to promote positive mental health, according to some embodiments of the present disclosure are described.

A method for improving the mental health of children, families and homes using “positive feedback loop” technology is described. One or more aspects of the method include obtaining a device; training the device to assign a predetermined threshold to at least one input, where the at least one input comprises a measurement of at least one condition of an ambient environment; measuring the at least one input; assigning a score to the measurement of the at least one condition of an ambient environment; evaluating whether the score exceeds the predetermined threshold; alerting at least one user when the score exceeds the predetermined threshold; and repeating the measuring, assigning, and evaluating steps until the score exceeds the predetermined threshold when the score does not exceed the predetermined threshold.

A non-limiting example of a particular method for improving the mental health of children, families and homes using “positive feedback loop” technology may include, but is not limited to, the following method: obtaining a device, where the device is configured to be operated by at least one user; training the device to assign a predetermined threshold to at least one audio test input, where the at least one audio test input comprises at least one audio test measurement of an ambient environment, where the at least one audio test measurement is selected from the group consisting of: a decibel level, at least one speech characteristic or any combination thereof, and where the predetermined threshold is selected from the group consisting of a maximum decibel level, at least one prohibited speech characteristic or any combination thereof; measuring at least one audio sample input, to obtain at least one audio sample measurement, where the at least one audio sample measurement is selected from the group consisting of: a current decibel level, at least one current speech characteristic or any combination thereof; assigning a score to the at least one audio sample measurement; evaluating whether the score exceeds the predetermined threshold, and: when the score exceeds a predetermined threshold, performing, with the device, at least one ameliorative action; and when the score does not exceed the predetermined threshold, repeating the measuring, assigning, and evaluating steps until the score exceeds the predetermined threshold.

A device for improving the mental health of children, families and homes using “positive feedback loop” technology is also described. One or more aspects of the device include at least one sensor and a processor. The at least one sensor can be configured to measure at least one input, where the at least one input comprises a measurement of at least one condition of an ambient environment and where the ambient environment comprises at least one user. The processor can be configured to train the device to assign a predetermined threshold to the at least one input, assign a score to the measurement of the at least one condition of an ambient environment, evaluate whether the score exceeds a predetermined threshold, alert the at least one user when the score exceeds the predetermined threshold, and not alert the at least one user when the score does not exceed the predetermined threshold.

### BRIEF DESCRIPTION OF THE DRAWINGS

FIGS. 1, 2A, 2B, 3, and 4 show examples of a mental health assistant device according to aspects of the present disclosure.

FIG. 5 shows an example of a method of improving mental health using “positive feedback loop” technology according to aspects of the present disclosure.

FIG. 6 shows an example of a process for training a mental health assistant device to utilize “positive feedback loop” technology according to aspects of the present disclosure.

### DETAILED DESCRIPTION

A method for improving the mental health of children, families and homes using “positive feedback loop” technology is described. As used herein a “positive feedback loop” is a set of software operations that adaptively respond to a user's positive emotions by increasing content giving rise to those positive emotions. In addition, a “positive feedback loop” is a set of software operations that adaptively respond to a user's negative emotions by decreasing content giving rise to those negative emotions.

As used herein, a “test input” is any input that is used to train a device described herein.

As used herein, a “test measurement” is a measurement within the test input that is used to train a device described herein.

As used herein, “an ameliorative action” is any action taken by the device in response to the predetermined threshold being exceeded. The “ameliorative action” may be ameliorative in the conventional sense of the word, but the ameliorative action may be any action performed by the device in response to the predetermined threshold being exceeded. Such an action may only consist of a prompt or alert, for example, in some non-limiting cases.

As used herein, a “maximum value” or “maximum level” is a value associated with a predetermined threshold (described further below). When a sample measurement exceeds this maximum value, the predetermined threshold has been exceeded, which may trigger an ameliorative action.

As used herein, a “prohibited” characteristic is a characteristic associated with a prohibited characteristic, the predetermined threshold has effectively been exceeded, which may trigger an ameliorative action. When the measurement is an audio measurement, for example, the characteristic may be a speech characteristic (e.g., speech content), and the prohibited characteristic may be a swear word.

As used herein, a “sample input” is any input that is used to determine whether there are audible distress signals in the environment.

As used herein, a “sample measurement” is any measurement within the sample input.

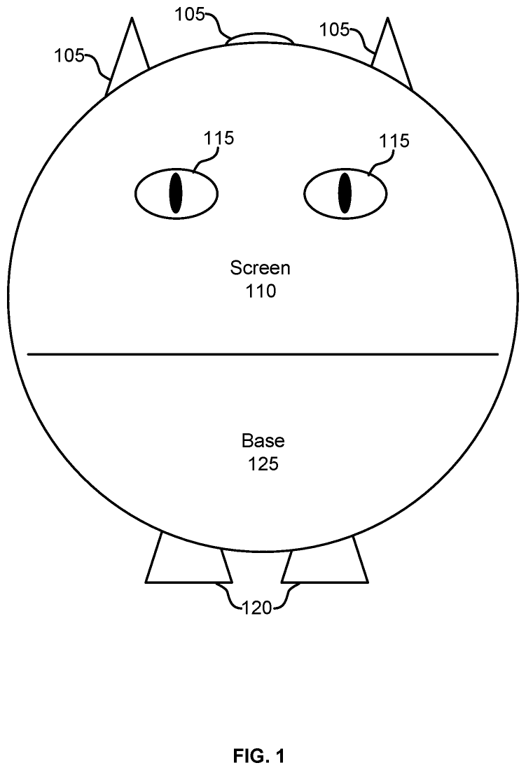

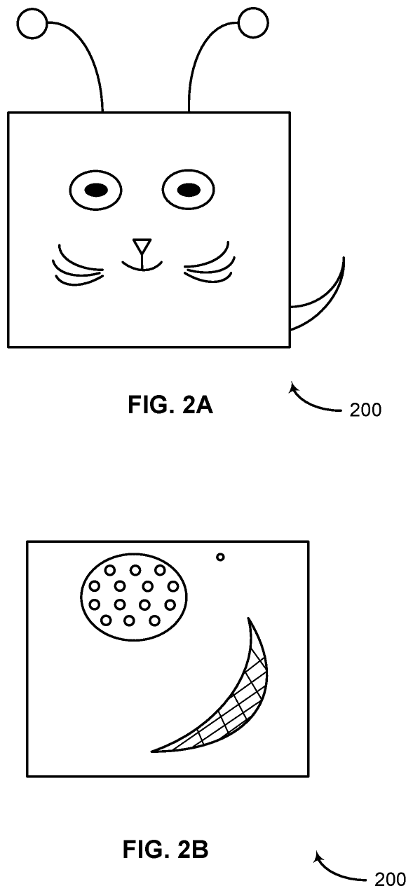

FIG. 1 shows a non-limiting example of a device 100 (e.g., a mental health assistant device) according to aspects of the present disclosure. Device 100 is an example of, or includes aspects of, the corresponding element described with reference to FIGS. 2-4. In one exemplary aspect, device 100 includes input components 105, and screen 110.

In some embodiments, the device 100, or any of the other exemplary devices or methods described herein may be used for Cognitive Behavioral Therapy (“CBT”), neuroplasticity training, emotional swing analysis, temper control, argument recognition and notification, addiction aid and warning notifications, calming support, emotional state and stress recognition, elder care, couple support, child emotion support, or any combination thereof.

In some examples, the device 100 may be round, square, or any other suitable shape. In some examples, a top half of the device 100 may comprise sounds, lights, a microphone, or any combination thereof. In some examples, the bottom half can be used for mechanics, power. While the top of the device 100 can be changed based on personal taste, in some examples, the top sufficiently porous to allow light and sounds are viewable and so ambient noise can be captured by an internal microphone. In some examples, the top half of the device 100 may light up with colors/patterns to signal/aid child, couple, adult, elder, parent, healthcare provider, or authority to determine mental mood/emotional state of those being monitored.

The screen 110 may comprise a monitor coupled with an integrated display, an integrated display (e.g., an LCD display), or any other screen 100 configured to display associated data or processing information. In some examples, output devices 100 other than the screen 110 can be used, such as printers, other computers or data storage devices 100, and computer networks. The screen 110 may be provided as a stand-alone device 100 or integrated with other elements of the device 100. For example, the screen 110 may include a touchscreen or touch-sensitive display. In such circumstances, a user input interface may be integrated with, or combined, with screen 110. In some embodiments, the screen 110 may include one or more of a monitor, a television, a liquid crystal display (LCD) for a mobile device 100, amorphous silicon display, low temperature poly silicon display, electronic ink display, electrophoretic display, active matrix display, electro-wetting display, electrofluidic display, cathode ray tube display, light emitting diode display, electroluminescent display, plasma display panel, high-performance addressing display, thin-film transistor display, organic light emitting diode display, surface conduction electron-emitter display (SED), laser television, carbon nanotubes, quantum dot display, interferometric modulator display, or any other suitable equipment for displaying visual images. In some embodiments, screen 110 may be HDTV-capable. In some embodiments, screen 110 may be a 3D display, and an interactive media guidance application and any suitable content may be displayed in 3D. A video card or graphics card may generate the output to the screen 110. The video card may offer various functions such as accelerated rendering of 3D scenes and 2D graphics, MPEG-2/MPEG-4 decoding, TV output, or the ability to connect multiple monitors. The video card may be any processing circuitry described above in relation to control circuitry. In some examples, the video card may be integrated with the control circuitry. In some examples, the screen 110 may include at least one light emitting diode (LED), where the at least one light emitting diode is configured to emit light having at least one color. The colored LED lighting may provide a calming effect if desired by the at least one user. In some examples, the screen 110 is configured to project shapes, colors, emotional faces, scenery, or any combination thereof.

Input components 105 may include any combination of devices that allow users to input information into the device 100, such as buttons, a keyboard, switches, and/or dials. Additionally or alternatively, the input component 105 may include a touch screen digitizer overlaid onto the screen 110 that can sense touch and interact with the screen 110. In some aspects, input components 105 may include a power button, a volume button, a help button, buttons for feedback, training, and microphone utilization, etc. In some cases, input components 105 may have different textures indicating different functionality of different buttons of the device 100. In some examples, speakers may be integrated with elements of device 100 (e.g., or speakers may be stand-alone units connected with device 100). In some aspects, the audio component of videos and other content displayed on screen 110 may be played through speakers. In some embodiments, the audio may be distributed to a receiver (not shown), which processes and outputs the audio via speakers.

In some cases, input components 105 may include one or more haptic feedback components. For instance, haptic feedback systems interact with a user's sense of touch by applying mechanical forces, vibrations, or motions. Haptic stimulation can be used to create or interact with virtual objects in a computer simulation, and to enhance the remote control of the device 100. Haptic devices may incorporate tactile sensors that measure forces exerted by the at least one user on the interface.

The device 100 may further comprise eyes 115, feet 120, or a combination thereof. Such components may provide the device 100 with the appearance of a pet. The device may also comprise a base 125. In some aspects, the base 125 comprises a plastic material. In some examples, the base 125 may comprise a power supply, circuitry, a memory, projection screen, vibration, a speaker, or a combination thereof. In some embodiments, the device 100 may be modified for sensory issues or enjoyment without departing from the scope of the present disclosure. In some embodiments, the device 100 may also have additional functions or components, such as but not limited to, notetaking capability, text to speech software for the auditorily impaired, guided meditation, braille on at least one surface of the device for the visually impaired, shape memory material (to adjust a texture or feel of the device 100 based on user feedback), compartments (to store emergency items, such as but limited to inhalers or epi-pens) additional components or functions, or any combination thereof.

FIGS. 2A and 2B show front and back views of a further example device 200 (e.g., a mental health assistant device) according to aspects of the present disclosure. Device 200 is an example of, or includes aspects of, the corresponding element described with reference to FIGS. 1, 3, and 4. As shown, the device 200 can have a variety of decorative elements. The device 200 may also have the look, feel, and/or touch of a pet, thus providing an additional emotionally supportive element.

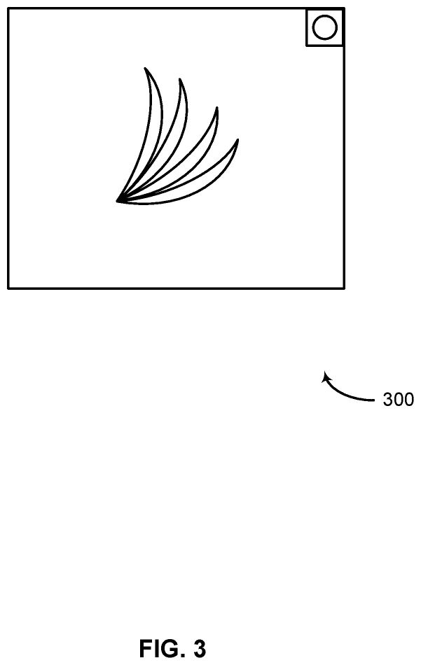

FIG. 3 shows an additional example of a device 300 (e.g., a mental health assistant apparatus) according to aspects of the present disclosure. Device 300 is an example of, or includes aspects of, the corresponding element described with reference to FIGS. 1, 2A, 2B, and 4.

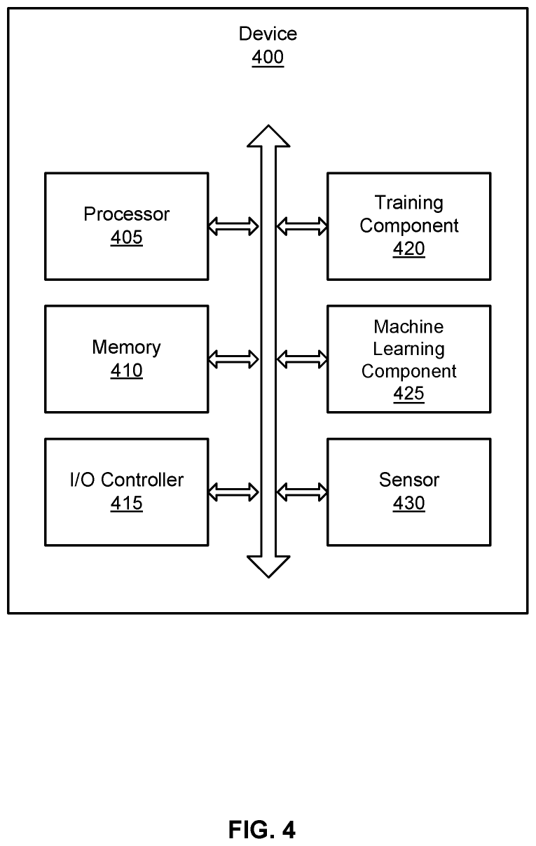

FIG. 4 depicts exemplary components of a device 400 (e.g., a mental health assistant apparatus) according to aspects of the present disclosure. In some aspects, device 400 is an example of, or includes aspects of, the corresponding element described with reference to FIGS. 1, 2A, 2B, and 3. In one aspect, device 400 includes processor 405, memory 410, I/O controller 415, training component 420, machine learning component 425, and at least one sensor 430.

In some aspects, the device 400 is portable. In some aspects, the device 400 is handheld. In some examples, device 400 comprises a base, a power button, a volume button, a help button, a power source, circuitry, RAM memory 410, a screen, or some combination thereof (e.g., as described in more detail herein). Non-limiting examples of a portable device 400 are shown in FIGS. 1, 2A, 2B, and 3.

In some examples, the processor 405 is an intelligent hardware device, (e.g., a general-purpose processing component, a digital signal processor (DSP), a central processing unit (CPU), a graphics processing unit (GPU), a microcontroller, an application specific integrated circuit (ASIC), a field programmable gate array (FPGA), a programmable logic device, a discrete gate or transistor logic component, a discrete hardware component, or any combination thereof). In some cases, the processor 405 is configured to operate memory 410 (e.g., a memory array) using a memory controller. In other cases, a memory controller is integrated into the processor 405. In some cases, the processor 405 is configured to execute computer-readable instructions stored in a memory 410 to perform various functions. In some embodiments, a processor 405 includes special-purpose components for modem processing, baseband processing, digital signal processing, or transmission processing.

According to some aspects, training component 420 is configured to train the device 400 to assign a predetermined threshold the at least one input, where the at least one input comprises a measurement of at least one condition of an ambient environment. As used herein “at least one condition of the ambient environment” is defined as any stimulus that does not originate from the device (e.g., device 400). In some examples, the stimulus may originate from the at least one user. In some examples, the stimulus may originate from the at least one user's surroundings. In some examples, the stimulus may originate from an individual in proximity with the at least one user. As used herein, a “measurement” is a quantitative assessment of the at least one user's surroundings, as measured by the device (e.g., device 400). In some examples a measurement or a plurality of measurements can be made using at least one sensor on the device (e.g., sensor 430). In some aspects, the measurement of the at least one condition of the ambient environment includes: a sound (e.g., decibel) level, a brightness level, a vibration level, a heat or temperature level, at least one speech characteristic, at least one global positioning system (GPS) coordinate, an electronic communication, a stress level, a breathing pattern of the at least one user, a brainwave pattern of the at least one user, an identity of the at least one user, or any combination thereof.

In some aspects, the heat or temperature level is indicative of body heat from the at least one user. A change in heat or temperature level may indicate that the at least one user is becoming agitated or, in some cases, is catching a fever. The heat or temperature level may be measured by a suitable heat or temperature sensor (e.g., a thermocouple) that may be present on the device 400.

In some aspects, the at least one speech characteristic includes: a quantity of swear words uttered by the at least one user, a quantity of slurred words uttered by the at least one user, a quantity of words indicating suicidal ideation uttered by the at least one user, a pitch of the speech, a tone of the speech, a timbre of the speech, a change in volume of a voice of the at least one user, or any combination thereof. A change in the at least one speech characteristic may indicate that the at least one user is impaired, under distress.

In some aspects, the GPS coordinate is of a predetermined location, where the predetermined location is input by the at least one user. In some aspects, the electronic communication is to at least one person to whom the at least one user normally does not wish to communicate. This person may be somebody who the at least one user considers to be a “bad-influence” or who provides the at least one user with undue emotional distress.

In some aspects, the stress level is determined by measuring a heart rate of the at least one user. In some aspects, the stress level is determined by measuring brain activity, perspiration, hormone levels, or any combination thereof.

In some aspects, the identity of the at least one user is determined using fingerprint analysis, facial recognition, voice recognition, or any combination thereof. In addition, the device 400 may respond to various changes to a user's vocal volume with responses of lights and sounds such as happy, sad, or celebratory.

In some examples, the brainwave pattern, stress level, breathing pattern, or another other biometric input may be used to detect medical issues. For instance, in some aspects, the brainwave pattern of the at least one user may be used to detect seizures.

The processor 405 may be configured to assign a score to the measurement of the at least one condition of an ambient environment and evaluate whether the score exceeds a predetermined threshold. The predetermined threshold may be determined by the at least one user or automatically using default settings of the device 400.

As used herein, the predetermined threshold quantifies a distress tolerance of at least one user of the device in response to the at least one input. The predetermined threshold for a given input may be determined manually (i.e., by the at least one user or the manufacturer) or automatically (using modeling such as via machine learning as described below), or a combination thereof.

In some examples. the predetermined threshold may be computed after a user is presented with a questionnaire which prompts the at least one user to quantify the at least one user's stress tolerance in various situations. The questionnaire, may in some instances, be interactive, such that the device 400 may generate or simulate stressful situations corresponding to the at least one input, the at least one condition of the ambient environment or combination thereof. In doing so, the device 400 may utilize, for example, sights, sounds, fragrances, textures, images, videos, lights or other stimuli and prompt the at least one user to input the severity of their response (e.g., on a scale from “1” to “10,” without limitation). In some examples, the severity of various outside stimuli and responses can be ranked based on pre-set scales of 0-10 (e.g., volume, words [0=“Sky”, 2=“Monday/work”, 9=“curse word”, 10=“suicide/hate”] pitch/tone/timbre, etc.). The pre-set scales may be determined by the at least one user by the manufacturer (as default settings), or a combination thereof (e.g., as default settings that may be adjusted by the user). Once the scaled inputs are obtained, the device 400 can then average the scaled inputs (e.g., via a weighted average, a geometric average, an arithmetic average, or other suitable average) to determine the predetermined threshold. In some examples, the predetermined threshold of the device 400, may later be iteratively optimized based on user feedback (as discussed below). In some instances, this initial process of providing the predetermined threshold could be performed by the at least one user prior to operation of the device. In some embodiments, this initial process of providing the predetermined threshold may be referred to as “training” the device. The “training” of the device could be made fun and interactive, like the process of training a pet.

In some examples, during training, the device 400 instructs the at least one user to measure the at least one input by displaying a prompt on the device 400, where the prompt includes instructions to measure the at least one input. In some examples, during training, the device 400 instructs the at least one user to assign a predetermined threshold to the at least one input by displaying a prompt on the device 400, where the prompt includes instructions to assign a predetermined threshold to the at least one input. In some examples, during training, the device 400 instructs the at least one user to assign a predetermined threshold to the at least one input by progressively increasing a magnitude of the measurement until the at least one user indicates that the magnitude of the measurement would exceed the predetermined threshold.

In some examples, the device 400 prompts the at least one user to adjust the predetermined threshold. In some aspects, prompting of the at least one user to adjust the predetermined threshold includes retraining the device 400. In some aspects, the device 400 is retrained by re-presenting the at least one input to at least one user, re-instructing the at least one user to measure the at least one input to thereby obtain the measurement of the at least one input, re-instructing the at least one user to assign a different predetermined threshold to the measurement of the at least one input.

In some aspects, prompting the at least one user to adjust the predetermined threshold includes supplementing training of the device 400. In some aspects, supplementing the training of the device 400 includes presenting at least one additional input to at least one user, instructing the at least one user to measure the at least one additional input to thereby obtain the measurement of the at least one additional input, and instructing the at least one user to assign a predetermined threshold to the measurement of the at least one additional input.

In some examples, after a sufficient number of inputs have been processed by the device 400, the device 400 may be able to generate a model (e.g., a machine learning model as described herein), where the model is able to predict a response of the at least one user to additional inputs, even when those inputs have never been measured (i.e., a “predictive model.”) For instance, if the device 400 has processed, a response of the at least one user to strong smells, loud noises, rough textures, and bright lights, the device 400 may not require any additional measurement to determine a response of the at least one user to spicy foods. In some examples, the training step is performed until a sufficient number of inputs have been processed by the device 400 for the device 400 to generate the predictive model. When the predictive model has been generated, the device 400 may prompt the user to either continue the training step (for improved accuracy) or to use the device 400. Regarding the “sufficient number of inputs,” the particular amount is not particularly limited and may vary based on the device 400, the types of inputs measured, the similarity of the inputs to each other, the number of measurements required to obtain an accurate data set, or other relevant factors.

In some aspects, the device 400 is configured to allow at least two users to communicate with one another. In some aspects, the communication includes audio communication, video communication, text communication or any combination thereof. In some aspects, the device 400 is trained by the at least one user. In some aspects, the device 400 is trained by a set of users. In some aspects, the at least one user of the set of users is a pet. In some aspects, the at least one user holds the pet during training.

In some examples, the processor 405 may initiate at least one ameliorative action when the score exceeds the predetermined threshold. For instance, the processor 405 may alert the at least one user when the score exceeds the predetermined threshold and does not alert the at least one user when the score does not exceed the predetermined threshold. In some examples, the alert signifies that the environmental stress level has exceeded the at least one user's stress tolerance based on training data stored within the device 400.

In some examples, device 400 prompts the at least one user to dismiss the alert. Dismissal of the alert may indicate that the at least one user's stress tolerance has not been reached, contrary to the training data stored within the device 400. This may indicate that the predetermined threshold needs to be further optimized. Accordingly, in some aspects, the at least one user will not be alerted when an identical score is assigned to an identical measurement of an identical type of the at least one condition upon adjustment of at least one of: the score, the predetermined threshold, or any combination thereof. In some instances, dismissing the alert may prompt the processor 405 to instruct the at least one user to repeat some or all of the training of the device 400. In some instances, dismissing the alert may prompt the processor 405 to instruct the at least one user to input a new scaled input corresponding to the dismissed alert.

In some aspects, the at least one ameliorative action includes: an alert to a police department, an alert to at least one medical provider, playing music, lowering a temperature of at least one room, altering lighting of at least one room, lowering volume on the device 400, emitting a fragrance (e.g., an essential oil or a perfume), playing a mantra or any combination thereof. In some aspects, the at least one ameliorative action is not performed automatically and is only performed after the at least one corrective action is manually initiated by the at least one user.

In some examples, the alert is tailored to specific characteristics of the at least one user. For instance, if the at least one user is asthmatic, the device 400 may emit a soothing fragrance. If the at least one user is epileptic, the device may alert the at least one user's surroundings or obtain medical attention. If the at least one user is prone to anxiety, the device may initiate a meditation. In some embodiments, the tailored alerts are generated using training data obtained from training the device.

According to some aspects, the processor 405 assigns a score to the measurement of the at least one condition of an ambient environment. In some examples, the processor 405 evaluates whether the score exceeds the predetermined threshold. In some examples, processor 405 alerts at least one user when the score exceeds the predetermined threshold. In some examples, processor 405 repeats the measuring, assigning, and evaluating steps until the score exceeds the predetermined threshold when the score does not exceed the predetermined threshold.

In some aspects, the predetermined threshold is determined by a manufacturer. In some aspects, the predetermined threshold is determined by training at least one machine learning (ML) or artificial intelligence (AI) algorithm (as discussed in more detail below). In some aspects, the predetermined threshold is proportional to a magnitude of the measurement, and where the magnitude of the measurement quantifies an extent of environmental distress associated with the measurement. In some aspects, the predetermined threshold is determined by the at least one user prior to the measuring step. In some aspects, the predetermined threshold is determined by a manufacturer.

In some examples, processor 405 performs the ameliorative action by alerting the at least one user, by emitting at least one frequency, emitting at least one sound, emitting at least one fragrance, emitting at least one light, sending at least one notification, or any combination thereof. In some examples, the at least one sound may include music, laughter, soothing sounds, or familiar voices (such as the voice of a movie or television character). In some examples, additional sounds may be downloadable by the device 400 or using at least one separate device. In some aspects, the emitting of the at least one frequency includes: emitting at least one light, initiating at least one vibration, displaying at least one image, or any combination thereof. In some aspects, the at least one sound includes: music, an alarm, a pitch, a tone, a mantra, a voice, or any combination thereof. In some aspects, the at least one notification includes: a text, a phone call, an email, a voicemail, an alert to a police department, an alert to an emergency department, an alert to at least one medical provider, or any combination thereof.

In some examples, processor 405 assigns an individual score to each of the set of measurements, thereby obtaining a set of individual scores. In some examples, processor 405 determines an aggregate score from the set of individual scores, where the score assigned to the measurement of the at least one condition of the ambient environment is the aggregate score. In some examples, processor 405 evaluates whether the aggregate score exceeds a predetermined threshold. In some examples, processor 405 alerts the at least one user when the aggregate score exceeds a predetermined threshold. In some examples, processor 405 evaluates steps until the aggregate score exceeds the predetermined threshold when the aggregate score does not exceed the predetermined threshold. In some aspects, the aggregate score from the set of individual scores is determined by calculating an arithmetic average of each of the set of individual scores, calculating a geometric average of each of the set of individual scores, inputting the set of individual scores into the at least one ML or AI algorithm, or any combination thereof.

The memory 410 in may include random access memory (RAM), read-only memory (ROM), or a hard disk. Examples of memory 410 also include solid state memory and a hard disk drive. In some examples, the memory is used to store computer-readable, computer-executable software including instructions that, when executed, cause a processor to perform various functions described herein. In some cases, the memory contains, among other things, a basic input/output system (BIOS) which controls basic hardware or software operation such as the interaction with peripheral components or memory 410. In some cases, a memory controller operates memory cells. For example, the memory controller can include a row decoder, column decoder, or both. In some cases, memory cells within a memory store information in the form of a logical state.

The I/O controller 415 may manage input and output signals for device 400. I/O controller 415 may also manage peripherals not integrated into device 400. In some cases, an I/O controller 415 may represent a physical connection or port to an external peripheral. In some cases, an I/O controller 415 may utilize an operating system such as iOS®, ANDROID®, MS-DOS®, MS-WINDOWS®, OS/2®, UNIX®, LINUX®, or another known operating system. In other cases, an I/O controller 415 may represent or interact with a modem, a keyboard, a mouse, a touchscreen, or a similar apparatus. In some cases, an I/O controller 415 may be implemented as part of a processor. In some cases, a user may interact with device 400 via I/O controller 415 or via hardware components controlled by an I/O controller 415.

According to some aspects, training component 420 trains the device 400 to assign a predetermined threshold to at least one input, where the at least one input includes a measurement of at least one condition of an ambient environment. In some aspects, the at least one input is a set of inputs. In some examples the device 400 may be trained manually (e.g., by using a button on the device 400 or using software installed on the device), remotely (over the internet or using an external device such as a phone, computer, or tablet), or a combination thereof.

In some examples, training component 420 trains the at least one ML or AI algorithm to adjust at least one of: the score, the predetermined threshold, or any combination thereof, upon the at least one user dismissing the alert. In some aspects, the measurement of at least one condition of an ambient environment includes a set of measurements, where each of the set of measurements corresponds to a condition of the ambient environment. In some aspects, the device 400 is trained using at least one ML algorithm, at least one AI algorithm, or any combination thereof. In some examples, the raining component 420 presents the at least one input to at least one user. In some examples, the training component 420 instructs the at least one user to measure the at least one input to thereby obtain the measurement of the at least one input. In some examples, training component 420 instructs the at least one user to assign a predetermined threshold to the measurement of the at least one input. In some aspects, the training of the device 400 includes storing the predetermined threshold in a memory 410 of the device 400. In some aspects, the magnitude of the measurement is progressively increased by prompting the at least one user to progressively increase the magnitude of the at least one input. In some aspects, the magnitude of the measurement is progressively increased by playing back the measurement at a progressively increasing volume. In some aspects, the training component 420 may include an artificial neural network (ANN), a convolutional neural network (CNN), a recurrent neural network (RNN), an LSTM, or a neural processing unit (NPU).

The training component 420 may include a machine learning component 425. In some examples, the machine learning component 425 may include, or may implement aspects of, an artificial neural network (ANN). An ANN is a hardware or a software component that includes a number of connected nodes (i.e., artificial neurons), which loosely correspond to the neurons in a human brain. Each connection, or edge, transmits a signal from one node to another (like the physical synapses in a brain). When a node receives a signal, it processes the signal and then transmits the processed signal to other connected nodes. In some cases, the signals between nodes comprise real numbers, and the output of each node is computed by a function of the sum of its inputs. Each node and edge may be associated with one or more node weights that determine how the signal is processed and transmitted. During the training process, these weights may be adjusted (e.g., via training component 420) to improve the accuracy of the result (i.e., by minimizing a loss function which corresponds in some way to the difference between the current result and the target result). The weight of an edge increases or decreases the strength of the signal transmitted between nodes. In some cases, nodes have a threshold below which a signal is not transmitted at all. In some examples, the nodes are aggregated into layers. Different layers perform different transformations on their inputs. The initial layer is known as the input layer and the last layer is known as the output layer. In some cases, signals traverse certain layers multiple times.

In some examples, the machine learning component 425 may include, or may implement aspects of, a convolutional neural network (CNN). A CNN is a class of neural network that is commonly used in computer vision or image classification systems. In some cases, a CNN may enable processing of digital images with minimal pre-processing. A CNN may be characterized by the use of convolutional (or cross-correlational) hidden layers. These layers apply a convolution operation to the input before signaling the result to the next layer. Each convolutional node may process data for a limited field of input (i.e., the receptive field). During a forward pass of the CNN, filters at each layer may be convolved across the input volume, computing the dot product between the filter and the input. During the training process, the filters may be modified so that they activate when they detect a particular feature within the input.

In some examples, the machine learning component 425 may include, or may implement aspects of, a recurrent neural network (RNN). A RNN is a class of ANN in which connections between nodes form a directed graph along an ordered (i.e., a temporal) sequence. This enables an RNN to model temporally dynamic behavior such as predicting what element should come next in a sequence. Thus, an RNN is suitable for tasks that involve ordered sequences such as text recognition (where words are ordered in a sentence). The term RNN may include finite impulse recurrent networks (characterized by nodes forming a directed acyclic graph), and infinite impulse recurrent networks (characterized by nodes forming a directed cyclic graph).

In some examples, machine learning component 425 may include, or may implement aspects of, a long short-term memory (LSTM) network. A LSTM is a form of RNN that includes feedback connections. In one example, and LSTM includes a cell, an input gate, an output gate and a forget gate. The cell stores values for a certain amount of time, and the gates dictate the flow of information into and out of the cell. LSTM networks may be used for making predictions based on series data where there can be gaps of unknown size between related information in the series. LSTMs can help mitigate the vanishing gradient (and exploding gradient) problems when training an RNN. For instance, the machine learning component 425 may include a neural processing unit (NPU) capable of implementing a neural network described herein. An NPU is a microprocessor that specializes in the acceleration of machine learning algorithms. For example, an NPU may operate on predictive models such as ANNs or random forests (RFs). In some cases, an NPU is designed in a way that makes it unsuitable for general purpose computing such as that performed by a CPU. Additionally or alternatively, the software support for an NPU may not be developed for general purpose computing.

In some examples, machine learning component 425 comprises a machine learning model generated by the training component 420.

According to some aspects, the at least one sensor 430 is configured to measure the at least one input, where the at least one input comprises a measurement of at least one condition of the ambient environment where the at least one user is located. In some aspects, the at least one sensor 430 includes: a microphone, a thermocouple, an infrared (IR) sensor, a touch screen, an accelerometer, a camera, a barometer, a fingerprint reader, or any combination thereof. In some aspects, the at least one input includes: a sound input, a light input, a haptic input, a temperature input, a pressure input (e.g., water pressure to indicate if the user is drowning), a speed input (e.g., driving speed, an indication that the user is running), a heat input, a linguistic input, a location input, a chemical input (e.g., alcohol, drugs, blood sugar), a biometric input or any combination thereof. In some examples, the device 400 comprises a plurality of sensors 430.

In one non-limiting example, the at least one sensor 430 may be an optical instrument (e.g., an image sensor, camera, etc.) for recording or capturing images, which may be stored locally, transmitted to another location, etc. For example, an image sensor 430 may capture visual information using one or more photosensitive elements that may be tuned for sensitivity to a visible spectrum of electromagnetic radiation. The resolution of such visual information may be measured in pixels, where each pixel may relate an independent piece of captured information. In some cases, each pixel may thus correspond to one component of, for example, a two-dimensional (2D) Fourier transform of an image. Computation methods may use pixel information to reconstruct images captured by the device 400. In a camera, image sensors 430 may convert light incident on a camera lens into an analog or digital signal. An electronic device may then display an image on a display panel based on the digital signal.

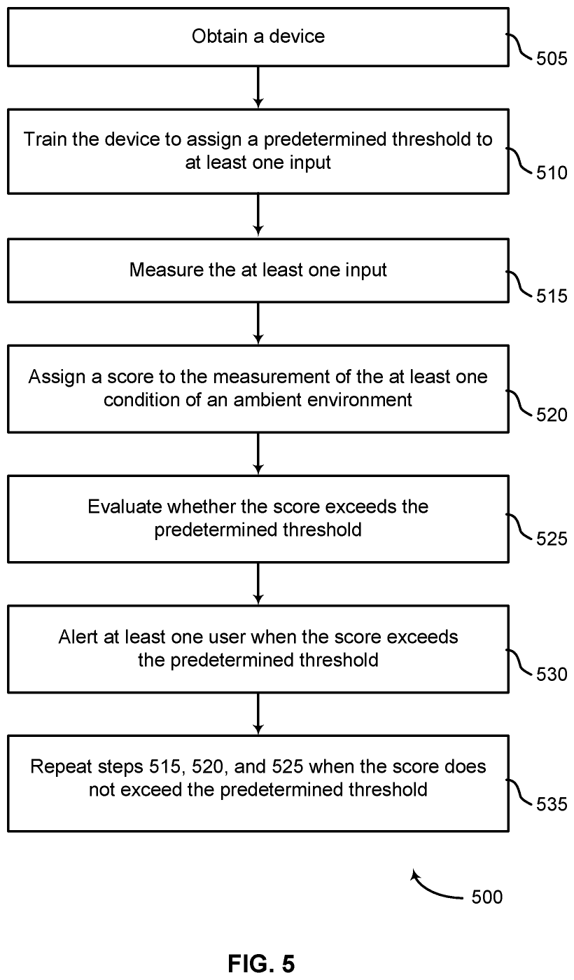

FIG. 5 shows an example of a method 500 of improving mental health according to at least some aspects of the present disclosure. In some examples, these operations are performed by a system including a processor executing a set of codes to control functional elements of an apparatus. Additionally or alternatively, certain processes are performed using special-purpose hardware. Generally, these operations are performed according to the methods and processes described in accordance with aspects of the present disclosure. In some cases, the operations described herein are composed of various substeps, or are performed in conjunction with other operations. At operation 505, at least one user obtains a device. In some cases, the operations of this step refer to, or may be performed by, a device as described with reference to FIGS. 1-4. At operation 510, the system trains the device to assign a predetermined threshold to at least one input, where the at least one input includes a measurement of at least one condition of an ambient environment. In some cases, the operations of this step refer to, or may be performed by, a training component as described with reference to FIG. 4. At operation 515, the system measures the at least one input. In some cases, the operations of this step refer to, or may be performed by, at least one sensor as described with reference to FIG. 4. At operation 520, the system assigns a score to the measurement of the at least one condition of an ambient environment. In some cases, the operations of this step refer to, or may be performed by, a processor as described with reference to FIG. 4. At operation 525, the system evaluates whether the score exceeds the predetermined threshold. In some cases, the operations of this step refer to, or may be performed by, a processor as described with reference to FIG. 4. At operation 530, the system alerts at least one user when the score exceeds the predetermined threshold. In some cases, the operations of this step refer to, or may be performed by, a processor as described with reference to FIG. 4. At operation 535, the system repeats the measuring, assigning, and evaluating steps until the score exceeds the predetermined threshold when the score does not exceed the predetermined threshold. In some cases, the operations of this step refer to, or may be performed by, a processor as described with reference to FIG. 4.

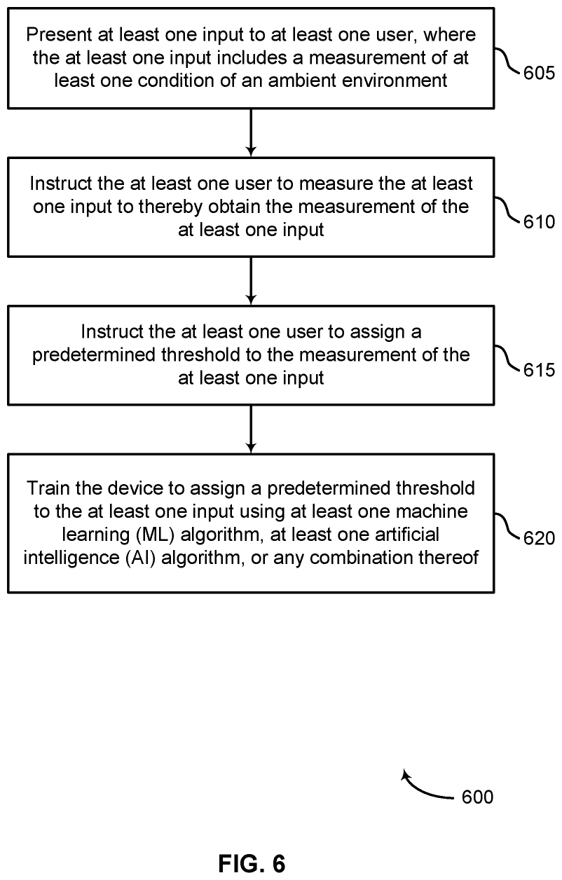

FIG. 6 shows an example of 600 FIG. 6 shows an example of a method for training a mental health assistant device according to at least some aspects of the present disclosure. In some examples, these operations are performed by a system including a processor executing a set of codes to control functional elements of an apparatus. Additionally, or alternatively, certain processes are performed using special-purpose hardware. Generally, these operations are performed according to the methods and processes described in accordance with aspects of the present disclosure. In some cases, the operations described herein are composed of various substeps, or are performed in conjunction with other operations. At operation 605, the system presents at least one input to at least one user, where the at least one input includes a measurement of at least one condition of an ambient environment. In some cases, the operations of this step refer to, or may be performed by, a training component as described with reference to FIG. 4. At operation 610, the system instructs the at least one user to measure the at least one input to thereby obtain the measurement of the at least one input. In some cases, the operations of this step refer to, or may be performed by, a training component as described with reference to FIG. 4. At operation 615, the system instructs the at least one user to assign a predetermined threshold to the measurement of the at least one input. In some cases, the operations of this step refer to, or may be performed by, a training component as described with reference to FIG. 4. At operation 620, the system trains the device to assign a predetermined threshold to the at least one input using at least one ML algorithm, at least one AI algorithm, or any combination thereof. In some aspects, the machine learning algorithm includes a neural network.

The neural network may use reinforcement learning, supervised learning, or unsupervised learning. In some cases, the operations of this step refer to, or may be performed by, a training component as described with reference to FIG. 4. Reinforcement learning is one of three basic machine learning paradigms, alongside supervised learning and unsupervised learning. Specifically, reinforcement learning relates to how software agents make decisions in order to maximize a reward. The decision making model may be referred to as a policy. This type of learning differs from supervised learning in that labeled training data is not needed, and errors need not be explicitly corrected. Instead, reinforcement learning balances exploration of unknown options and exploitation of existing knowledge. In some cases, the reinforcement learning environment is stated in the form of a Markov decision process (MDP). Furthermore, many reinforcement learning algorithms utilize dynamic programming techniques. However, one difference between reinforcement learning and other dynamic programming methods is that reinforcement learning does not require an exact mathematical model of the MDP. Therefore, reinforcement learning models may be used for large MDPs where exact methods are impractical. Supervised learning is one of three basic machine learning paradigms, alongside unsupervised learning and reinforcement learning. Supervised learning is a machine learning technique based on learning a function that maps an input to an output based on example input-output pairs.

Supervised learning generates a function for predicting labeled data based on labeled training data consisting of a set of training examples. In some cases, each example is a pair consisting of an input object (typically a vector) and a desired output value (i.e., a single value, or an output vector). A supervised learning algorithm analyzes the training data and produces the inferred function, which can be used for mapping new examples. In some cases, the learning results in a function that correctly determines the class labels for unseen instances. In other words, the learning algorithm generalizes from the training data to unseen examples.

Unsupervised learning is one of three basic machine learning paradigms, alongside supervised learning and reinforcement learning. Unsupervised learning draws inferences from datasets consisting of input data without labeled responses. Unsupervised learning may be used to find hidden patterns or grouping in data. For example, cluster analysis is a form of unsupervised learning. Clusters may be identified using measures of similarity such as Euclidean or probabilistic distance.

For example, as described herein, a neural network (e.g., machine learning component 425) is a type of computer algorithm that is capable of learning specific patterns without being explicitly programmed, but through iterations over known data. A neural network may refer to a cognitive model that includes input nodes, hidden nodes, and output nodes. Nodes in the network may have an activation function that computes whether the node is activated based on the output of previous nodes. Training the device (e.g., mental health assistant device 400) may involve a training component (e.g., a training component 420) supplying values for the inputs, and modifying edge weights and activation functions (algorithmically or randomly) until the result approximates a set of desired outputs.

Some modifications to the disclosure may be readily apparent to those skilled in the art, and the principles defined herein may be applied to other variations without departing from the scope of the disclosure. Thus, the disclosure is not limited to the examples and designs described herein, but is to be accorded the broadest scope consistent with the principles and novel features disclosed herein.

The described methods may be implemented or performed by devices that include a general-purpose processor, a digital signal processor (DSP), an application specific integrated circuit (ASIC), a field programmable gate array (FPGA) or other programmable logic device, discrete gate or transistor logic, discrete hardware components, or any combination thereof. A general-purpose processor may be a microprocessor, a conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration). Thus, the functions described herein may be implemented in hardware or software and may be executed by a processor, firmware, or any combination thereof. If implemented in software executed by a processor, the functions may be stored in the form of instructions or code on a computer-readable medium.

Computer-readable media includes both non-transitory computer storage media and communication media including any medium that facilitates transfer of code or data. A non-transitory storage medium may be any available medium that can be accessed by a computer. For example, non-transitory computer-readable media can comprise RAM, ROM, electrically erasable programmable read-only memory (EEPROM), compact disc (CD) or other optical disk storage, magnetic disk storage, or any other non-transitory medium for carrying or storing data or code.

Also, connecting components may be properly termed computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

In this disclosure and the following claims, the word “or” indicates an inclusive list such that, for example, the list of X, Y, or Z means X or Y or Z or XY or XZ or YZ or XYZ. Also the phrase “based on” is not used to represent a closed set of conditions. For example, a step that is described as “based on condition A” may be based on both condition A and condition B. In other words, the phrase “based on” shall be construed to mean “based at least in part on.” Also, the words “a” or “an” indicate “at least one.”

The description and drawings described herein represent example configurations and do not represent all the implementations within the scope of the claims. For example, the operations and steps may be rearranged, combined or otherwise modified. Also, structures and devices may be represented in the form of block diagrams to represent the relationship between components and avoid obscuring the described concepts. Similar components or features may have the same name but may have different reference numbers corresponding to different figures.

Without being limiting, several exemplary non-limiting aspects of the present disclosure are provided below.

A method comprising:
obtaining a device;
training the device to assign a predetermined threshold to at least one input, where the at least one input comprises a measurement of at least one condition of an ambient environment;
measuring the at least one input;
assigning a score to the measurement of the at least one condition of an ambient environment;
evaluating whether the score exceeds the predetermined threshold, and:
   when the score exceeds a predetermined threshold, alerting at least one user; and
   when the score does not exceed the predetermined threshold, repeating the measuring, assigning, and evaluating steps until the score exceeds the predetermined threshold.

The method of paragraph [0083], where the at least one input comprises: a sound input, a light input, a haptic input, a temperature input, a pressure input, a speed input, a heat input, a linguistic input, a location input, a chemical input, a biometric input or any combination thereof.

The method of paragraph [0071], where the measurement of the at least one condition of the ambient environment comprises: a decibel level, a brightness level, a vibration level, a heat or temperature level, at least one speech characteristic, at least one global positioning system (GPS) coordinate, an electronic communication, a stress level, a breathing pattern of the at least one user, a brainwave pattern of the at least one user, an identity of the at least one user, or any combination thereof.

The method of paragraph [0085], where the heat or temperature level is indicative of body heat from the at least one user.

The method of paragraph [0085], where the at least one speech characteristic comprises: a quantity of swear words uttered by the at least one user, a quantity of slurred words uttered by the at least one user, a quantity of words indicating suicidal ideation uttered by the at least one user, a pitch of the speech, a tone of the speech, a timbre of the speech, a change in volume of a voice of the at least one user, or any combination thereof.

The method of paragraph [0085], where the GPS coordinate is of a predetermined location, where the predetermined location is input by the at least one user.

The method of paragraph [0085], where the electronic communication is to at least one person to whom the at least one user normally does not wish to communicate.

The method of paragraph [0085], where the stress level is determined by measuring a heart rate of the at least one user.

The method of paragraph [0085], where the identity of the at least one user is determined using fingerprint analysis, facial recognition, voice recognition, or any combination thereof.

The method of paragraph [0071], where the score is input by the at least one user prior to the measuring.

The method of paragraph [0071], where the predetermined threshold is determined by a manufacturer.

The method of paragraph [0071], where the predetermined threshold is determined by training at least one ML or AI algorithm.

The method of paragraph [0071], where the predetermined threshold is proportional to a magnitude of the measurement, and where the magnitude of the measurement quantifies an extent of environmental distress associated with the measurement.

The method of paragraph [0071], where the predetermined threshold is determined by the at least one user prior to the measuring step.

The method of paragraph [0071], where the predetermined threshold is determined by a manufacturer.

The method of paragraph [0071], where the predetermined threshold is determined by training at least one ML or AI algorithm.

The method of paragraph [0071], where alerting the at least one user comprises: emitting at least one frequency, emitting at least one sound, emitting at least one fragrance, emitting at least one light, sending at least one notification, or any combination thereof.

The method of paragraph [0099], where the emitting of the at least one frequency comprises: emitting at least one light, initiating at least one vibration, displaying at least one image, or any combination thereof.

The method of paragraph [0100], where the at least one sound comprises: music, an alarm, a pitch, a tone, a mantra, a voice, or any combination thereof.

The method of paragraph [0100], where the at least one notification comprises: a text, a phone call, an email, a voicemail, an alert to a police department, an alert to an emergency department, an alert to at least one medical provider, or any combination thereof.

The method of paragraph [0071], further comprising, after alerting the at least one user, prompting the at least one user to dismiss the alert.

The method of paragraph [0103], where upon the at least one user dismissing the alert, the method further comprising training at least one ML or AI algorithm to adjust at least one of: the score, the predetermined threshold, or any combination thereof.

The method of paragraph [0104], where upon an adjustment of at least one of: the score, the predetermined threshold, or any combination thereof, the at least one user will not be alerted when an identical score is assigned to an identical measurement of an identical type of the at least one condition.

The method of paragraph [0071], further comprising, after alerting the at least one user, prompting the at least one user to initiate at least one corrective action.

The method of paragraph [0106] where the at least one corrective action comprises: an alert to a police department, an alert to at least one medical provider, playing music, lowering a temperature of at least one room, altering lighting of at least one room, lowering volume on the device, emitting a fragrance, playing a mantra or any combination thereof.

The method of paragraph [0106], where the at least one corrective action is not performed automatically and is only performed after the at least one corrective action is initiated by the at least one user.

The method of paragraph [0071], where the at least one input is a plurality of inputs.

The method of paragraph [0109], where the measurement of at least one condition of an ambient environment comprises a plurality of measurements, where each of the plurality of measurements corresponds a condition of the ambient environment.

The method of paragraph [0110], where the score is an aggregate score, and where the method further comprises:
assigning an individual score to each of the plurality of measurements, thereby obtaining a plurality of individual scores; and
determining an aggregate score from the plurality of individual scores.

The method of paragraph [0111], further comprising:
evaluating whether the aggregate score exceeds a predetermined threshold, and:
   when the aggregate score exceeds a predetermined threshold, alerting the at least one user; and
   when the aggregate score does not exceed the predetermined threshold, repeating the obtaining, assigning, and evaluating steps until the aggregate score exceeds the predetermined threshold.

The method of paragraph [0111], where the determining an aggregate score from the plurality of individual scores comprises: calculating an arithmetic average of each of the plurality of individual scores, calculating a geometric average of each of the plurality of individual scores, inputting the plurality of individual scores into at least one ML or AI algorithm, or any combination thereof.

The method of paragraph [0071], where training the device is performed using at least one ML algorithm, at least one AI algorithm, or any combination thereof.

The method of paragraph [0071], where training the device comprises:
presenting the at least one input to at least one user;
instructing the at least one user to measure the at least one input to thereby obtain the measurement of the at least one input; and
instructing the at least one user to assign a predetermined threshold to the measurement of the at least one input.

The method of paragraph [0115], where training the device comprises storing the predetermined threshold in a memory of the device.

The method of paragraph [0115], where instructing the at least one user to measure the at least one input comprises displaying a prompt on the device, where the prompt comprises instructions to measure the at least one input.

The method of paragraph [0115], where instructing the at least one user to assign a predetermined threshold to the at least one input comprises displaying a prompt on the device, where the prompt comprises instructions to assign a predetermined threshold to the at least one input.

The method of paragraph [0115], where instructing the at least one user to assign a predetermined threshold to the at least one input comprises progressively increasing a magnitude of the measurement until the at least one user indicates that the magnitude of the measurement exceeds the predetermined threshold.

The method of paragraph [0119], where progressively increasing a magnitude of the measurement comprises prompting the at least one user to progressively increase the magnitude of the at least one input.

The method of paragraph [0119], where progressively increasing a magnitude of the measurement comprises playing back the measurement at a progressively increasing volume.

The method of paragraph [0071], where the device is configured to allow at least two users to communicate with one another.

The method of paragraph [0122], where the communication comprises audio communication, video communication, text communication or any combination thereof.

The method of paragraph [0071], where the device is trained by the at least one user.

The method of paragraph [0071], where the device is trained by a plurality of users.

The method of paragraph [0125], where at least one user of the plurality of users is a pet.

The method of paragraph [0126], where at least one user holds the pet during training.

A device comprising:
at least one sensor, where the at least one sensor is configured to measure at least one input, where the at least one input comprises a measurement of at least one condition of an ambient environment, where the ambient environment comprises at least one user;
at least one processor, where the at least one processor is configured to:
   train the device to assign a predetermined threshold to the at least one input;
   assign a score to the measurement of the at least one condition of an ambient environment;
   evaluate whether the score exceeds a predetermined threshold, and:
      when the score exceeds a predetermined threshold, the at least one processor is configured to alert the at least one user; and
      when the score does not exceed the predetermined threshold, the device is configured not to alert the at least one user.

The device of paragraph [0128], where the device is portable.

The device of paragraph [0128], where the at least one sensor comprises: a microphone, a thermocouple, an infrared (IR) sensor, a touch screen, an accelerometer, a camera, or any combination thereof.

The device of paragraph [0128], further comprising: a base, a power button, a volume button, a help button, a power source, circuitry, RAM, a screen, or any combination thereof.

The method of paragraph [0115], further comprising prompting the at least one user to adjust the predetermined threshold.

The method of paragraph [0132], where prompting the at least one user to adjust the predetermined threshold comprises retraining the device.

The method of paragraph [0133], where retraining the device comprises:
re-presenting the at least one input to at least one user;
re-instructing the at least one user to measure the at least one input to thereby obtain the measurement of the at least one input; and
re-instructing the at least one user to assign a different predetermined threshold to the measurement of the at least one input.

The method of paragraph [0133], where prompting the at least one user to adjust the predetermined threshold comprises supplementing training of the device.

The method of paragraph [0135] where supplementing training of the device comprises:
presenting at least one additional input to at least one user;
instructing the at least one user to measure the at least one additional input to thereby obtain the measurement of the at least one additional input; and
instructing the at least one user to assign a predetermined threshold to the measurement of the at least one additional input.

A method comprising:
obtaining a device, where the device comprises:
   a processor;
   a memory;
   a plurality of sensors; and
   a training component, where the training component comprises a neural network;
training the device, using the neural network, to obtain a machine learning model, where the machine learning model is configured to assign a predetermined threshold to at least one input, where the predetermined threshold quantifies a distress tolerance of at least one user of the device in response to the at least one input, and where the at least one input comprises a measurement of at least one condition of an ambient environment;
measuring the at least one input using at least one of the plurality of sensors to obtain the measurement of the at least one condition of the ambient environment;
assigning a score to the measurement of the at least one condition of the ambient environment;
evaluating, using the machine learning model, whether the score exceeds the predetermined threshold for the at least one input, and:
   when the score exceeds the predetermined threshold, alerting at least one user of the device; and
   when the score does not exceed the predetermined threshold, repeating the measuring, assigning, and evaluating steps until the score exceeds the predetermined threshold.

1. A method comprising:
obtaining a device, where the device is configured to be operated by at least one user;
training the device to assign a predetermined threshold to at least one audio test input, where the at least one audio test input comprises at least one audio test measurement of an ambient environment,
where the at least one audio test measurement is selected from the group consisting of: a decibel level, at least one speech characteristic or any combination thereof, and
where the predetermined threshold is selected from the group consisting of a maximum decibel level, at least one prohibited speech characteristic or any combination thereof;
measuring at least one audio sample input, to obtain at least one audio sample measurement, where the at least one audio sample measurement is selected from the group consisting of: a current decibel level, at least one current speech characteristic or any combination thereof
assigning a score to the at least one audio sample measurement;
evaluating whether the score exceeds the predetermined threshold, and:
when the score exceeds a predetermined threshold, performing, with the device, at least one ameliorative action; and
when the score does not exceed the predetermined threshold, repeating the measuring, assigning, and evaluating steps until the score exceeds the predetermined threshold.

2. The method of claim 1, where the at least one speech characteristic comprises: a quantity of swear words uttered by at least one user, a quantity of slurred words uttered by the at least one user, a quantity of words indicating suicidal ideation uttered by the at least one user, a pitch of the speech, a tone of the speech, a timbre of the speech, a change in volume of a voice of the at least one user, or any combination thereof.

3. The method of claim 1, where the score is input by the at least one user prior to the measuring.

4. The method of claim 1, where the predetermined threshold is determined by a manufacturer.

5. The method of claim 1, where the predetermined threshold is determined by training at least one ML or AI algorithm.

6. The method of claim 1, where the predetermined threshold is determined by the at least one user prior to the measuring step.

7. The method of claim 1, where the predetermined threshold is determined by a manufacturer.

8. The method of claim 1, where the predetermined threshold is determined by training at least one ML or AI algorithm.

9. The method of claim 1, where the at least one ameliorative action comprises: alerting the at least one user, emitting at least one frequency, emitting at least one sound, emitting at least one fragrance, emitting at least one light, sending at least one notification, or any combination thereof.

10. The method of claim 9, where the emitting of the at least one frequency comprises: emitting at least one light, initiating at least one vibration, displaying at least one image, or any combination thereof.

11. The method of claim 9, where the at least one sound comprises: music, an alarm, a pitch, a tone, a mantra, a voice, or any combination thereof.

12. The method of claim 9, where the at least one notification comprises: a text, a phone call, an email, a voicemail, an alert to a police department, an alert to an emergency department, an alert to at least one medical provider, or any combination thereof.

13. The method of claim 9, further comprising prompting the at least one user to dismiss the alert.

14. The method of claim 13, where upon the at least one user dismissing the alert, the method further comprising training at least one ML or AI algorithm to adjust at least one of: the score, the predetermined threshold, or any combination thereof.

15. The method of claim 14, where upon an adjustment of at least one of: the score, the predetermined threshold, or any combination thereof, the at least one user will not be alerted when an identical score is assigned to an identical measurement of an identical type.

16. The method of claim 1, where training the device comprises:
presenting the at least one input to the at least one user;
instructing the at least one user to measure the at least one audio input to obtain the at least one audio measurement; and
instructing the at least one user to assign a predetermined threshold to the at least one audio measurement.

17. The method of claim 16, where training the device comprises storing a record of the predetermined threshold in a memory of the device.

18. The method of claim 16, where instructing the at least one user to measure the at least one audio input comprises displaying a prompt on the device, where the prompt comprises instructions to measure the at least one audio input.

19. The method of claim 1, where the device is trained by the at least one user.

20. The method of claim 1, where the device comprises at least one sensor and at least one processor.
